(19) United States
(12) Patent Application Publication
(10) Pub. No.: US 2022/0193382 A1
(43) Pub. Date: Jun. 23, 2022

(54) System for Dermal Application of a Pharmaceutical and/or Cosmetic Composition

(71) Applicant: Dr. August Wolff GmbH & Co. KG Arzneimittel, Bielefeld (DE)

(72) Inventors: Thorsten CHRISTIANS, Bielefeld (DE); Ulrich KNIE, Bielefeld (DE); Christoph ABELS, Bielefeld (DE)

(21) Appl. No.: 17/602,211

(22) PCT Filed: Apr. 8, 2020
(86) PCT No.: PCT/EP2020/059991

§ 371 Date: Oct. 7, 2021

(30) Foreign Application Priority Data
Apr. 10, 2019 (EP) 19168474.5

(51) Int. Cl. A61M 35/00; A61K 31/40; A45D 40/28; A45D 34/04

(52) U.S. Cl. CPC A61M 35/003; A61K 31/40; A45D 2200/055; A45D 34/04; A45D 40/28

(57) ABSTRACT

The present invention relates to a system for applying and/or spreading a pharmaceutical and/or cosmetic composition on the skin. The system (100) comprises a container (110) for receiving a pharmaceutical and/or cosmetic composition (140); a dosing unit (120) mechanically coupled with the container (100) for dispensing a predetermined amount (141) of the composition (140) from the container (110) through an opening (121) of the dosing unit (120); and an applicator (130). The applicator (130) is configured to be temporarily coated or loaded with the predetermined amount (141) of the composition (140). Moreover, the applicator (130) is configured for spreading the predetermined amount (141) of the composition (140) on a skin area of a user. The applicator (130) is detachably connectable with the container (110) or the dosing unit (120).

The invention relates to systems for application of a pharmaceutical and/or a cosmetic composition on the skin of a subject, and more particularly for topical application of a pharmaceutical and/or a cosmetic composition on the skin of a subject.

Some pharmaceutical and/or cosmetic compositions must be applied (only) on particular skin areas by a user. For example, antiperspirant compositions are typically only applied on the skin of the armpit of a user. Because of certain active ingredients other skin areas of the user, for example, the fingers of the user, should not get into contact with the pharmaceutical and/or the cosmetic composition to avoid contamination. Moreover, some pharmaceutical and/or cosmetic compositions have to be applied with a well-defined dose to achieve the desired pharmaceutical and/or cosmetic effect and/or to avoid adverse effects.

Excessive sweating or hyperhidrosis is a condition in which sweating is beyond what is physiologically required to maintain normal thermal regulation of the human body. Thus, hyperhidrosis is an extremely inconvenient condition negatively affecting daily life of a person suffering therefrom. Hyperhidrosis can be divided into focal and general primary hyperhidrosis. The focal is bilaterally symmetrical: hands, feet, axillae or groins. Focal hyperhidrosis from the face/head does occur but is often part of the general form. Generalized sweating usually involves both the head and trunk and in severe cases also extremities and groins/glutes. Whereas the majority of patients affected have the primary form which is hereditary, there is also a secondary form, often related to an underlying disease.

A variety of treatments have been proposed for treating excessive sweating/hyperhidrosis which include aluminum containing antiperspirants (or deodorant), surgical removal of sweat glands and systemic or local treatment with anticholinergic compounds. Any of these treatments, however, exhibit sometimes serious disadvantages. Aluminum salts containing antiperspirants, in particular upon continuous use, stain or discolor the textiles coming into contact with the antiperspirant. In addition, the increasing amount of aluminum in waste water recently has also been increasing environmental concerns. On the other hand, removal of the sweat glands means surgery that may be accompanied by at least inconvenient or even serious side effects.

As stated above, anticholinergic glycopyrronium salts (also named glycopyrrolates) have been proposed for the treatment of excessive sweating and hyperhidrosis. WO 2006/069998 discloses the use of glycopyrronium bromide together with a large variety of other active ingredients for the treatment of excessive sweating. WO 2014/134510 relates to a specific glycopyrronium tosylate salt for the treatment of excessive sweating and hyperhidrosis.

For applying deodorant compositions, EP 2 571 394 B1 proposes a ‘deo’ roll-on comprising a dosing ball and a container containing liquid. The dosing ball continuously contacts the liquid provided in the container when the known deo roller is used. Hence, it is not possible to apply a predetermined dose of the deodorant composition on a skin area of the user.

Moreover, EP 1 793 936 B1 suggests a spraying apparatus for spraying a dose of a deodorant composition on a skin area in the armpit of the user. Spraying apparatus are only suitable for spraying very low viscous compositions. Moreover, users typically do not see the skin area on which the composition has to be sprayed. Hence, it may be difficult to apply the dose exactly to the intended skin area. Rather there is a significant risk to contaminate also other areas of the skin with the composition or even inhale the composition (may also affect any other persons next to the user) that may lead to undesired side effects.

Hence, it is an object of the present invention to provide a system configured for applying and/or spreading a predetermined dose of a pharmaceutical and/or cosmetic composition to a well-defined skin area of a user without contamination of the fingers/hands and/or other body parts of the user.

Said object has been addressed with the subject-matter of the independent claims. Advantageous embodiments are described in the dependent claims.

According to a first aspect of the present technique, it is proposed to have a system comprising a container for receiving or storing a pharmaceutical and/or cosmetic composition; a dosing unit mechanically coupled with the container for dispensing a predetermined amount of the composition from the container through an opening of the dosing unit; and an applicator. The applicator is configured to be temporarily coated or loaded, in particular wetted, with the predetermined amount of the composition. Further, the applicator is configured for applying and/or spreading the predetermined amount of the composition on a skin area of a user. The applicator is detachably connectable with the container or the dosing unit. For example, the applicator may be coupled to the dosing unit, which is mechanically coupled with the container. Alternatively, the applicator may be directly connectable with the container. In accordance with the present invention a composition can be applied to the skin of the user in a well-defined way, namely in an exactly defined amount to a specifically intended area of the skin without contamination of the fingers/hands and/or other body parts of the user that are not intended to be treated. In this way it is possible to safely apply and/or spread compositions being toxic upon topical administration above a specific amount or toxic when—unintentionally by way of contaminated hands orally (systemically) administered or when unintentionally administered to the eye. Because of being connectible the system is compact during after-use storage or during pre-use or post-use transportation. The risk of misplacing the applicator is reduced.

The phrase “temporarily coated” with respect to the applicator and like phases herein may mean loaded onto the applicator or received onto the applicator for being subsequently dispensed, for applying or spreading, onto a skin area of the user.

A part or surface of the applicator which is used for receiving the composition dispensed from the dosing unit and for applying or spreading the composition onto a skin area of the user, and a part of the applicator which is used for detachably connecting the applicator with the container or the dosing unit may be integrally formed i.e. may be formed as one-part, for example by molding.

According to a first embodiment of the system, the applicator may be washable. This may make it easier to keep the applicator in a hygienic condition.

In a second embodiment of the system, the applicator may be dishwasher safe. Providing a dishwasher safe applicator may reduce the effort required for cleaning the applicator. Accordingly, users may be more inclined to clean the applicator regularly thus increasing the hygiene of the applicator.

In a further embodiment of the system the applicator is temperature resistant up to a temperature of 59° C., in particular up to a temperature of 69° C., in particular up to a temperature of 85° C. A hotter environment may reduce the time for cleaning the applicator. In particular, a temperature resistance of up to a temperature of 59° C. may allow for using faster washing programs of dishwaters. A temperature resistance of up to a temperature of 69° C. may reduce the risk that the applicator becomes damaged when directly subjected to the warm water provided e.g. in the dishwasher. If the applicator is temperature resistant up to a temperature of 85° C., the applicator may be cleaned at temperatures which may further reduce the number of microorganisms on the surface of the applicator.

In an embodiment of the system, the applicator is detachably connectable with the container or the dosing unit at a first location of the container or a first location of the dosing unit. When connected at the first location of the container or at the first location of the dosing unit, the applicator acts as a closure or cap for the container. When the applicator acts as a closure for the container the dosing unit may be disposed in an inner space of the applicator.

In another embodiment, when the applicator acts as a closure for the container, the applicator is configured to seal the opening of the dosing unit. The applicator may comprise a sealing structure, such as a protrusion, extending into the inner space of the applicator and that is configured to contact the opening of the dosing unit to close or seal the opening of the dosing unit. Alternatively, the sealing structure may seal the opening of the dosing unit in a contact-less way i.e. without contacting the opening of the dosing unit, for example by surrounding or encasing the opening of the dosing unit. By sealing the opening of the dosing unit, a risk that a part of the composition leaks out of the dosing unit during transport of the system is at least partly obviated.

In another embodiment of the system, the applicator is detachably connectible to the container at a second location of the container such that the container forms an extension handle of the applicator.

According to another embodiment of the system, the applicator may be detachably connectable, particularly at the first locations or the second location, to the container or the dosing unit via a plug-in connection. Thus, the applicator may be connected to the container or the dosing unit with a simple translational movement.

In a further embodiment of the system, the applicator may be detachably connectable, particularly at the first locations or the second location, to the container or the dosing unit via a screw fitting, also referred to as screw and thread connection. A screw fitting may firmly connect the applicator and the container or the dosing unit.

In another embodiment of the system the applicator may be detachably connectable, particularly at the first locations or the second location, to the container or the dosing unit via a friction-fit connection. A friction-fit connection is easy to realize and may be operated by simple translation motion of the applicator relative to the dosing unit or the container.

In another embodiment of the system the applicator may be detachably connectable, particularly at the first locations or the second location, to the container or the dosing unit via a snap-on connection. A snap-on connection may provide direct feedback to the user when the applicator is securely held at the container or dosing unit. This may avoid that the applicator and container or dosing unit fall apart upon transportation of the system, for example, in a handbag.

According to another embodiment of the system, the applicator may be formed of a polymer. Manufacturing the applicator from a polymer may reduce the costs for the whole system. Moreover, the applicator of the system may be replaceable in case of damage or in case a new applicator is preferred due to hygienic or sanitary reasons.

In an embodiment of the system, the applicator comprises: a gripping part configured to be gripped by the user, and an application part configured to receive the composition dispensed through the opening of the dosing unit and to apply and/or spread on the skin of the user the composition so received. The application part has one of convex shape, a flat-top shape i.e. a flat shape or planar shape and a concave shape. Each of the shapes, i.e. the convex shape, the flat shape and the concave shape, are especially advantageous for application and spreading of the composition depending on a contour or characteristic of the skin area such as accessibility to the skin area, curvature of the skin area, expanse of the skin area, and so on and so forth.

In a preferred embodiment of the system, the applicator has a flat-top shape i.e. an entire top surface of the applicator forms the application part and is flat or planar i.e. not surrounded by any ridges or raised boundaries and may be without any curved region. In other words, the flat or planar shaped application part may extend as a flat plane or flat top of the applicator and then a surface of the applicator may bend downwards, from the flat plane, into the gripping part. In other words, the flat or planar shaped application part may extend as a flat plane or flat top of the applicator, and directly adjoining the flat plane or the flat top of the applicator is a surface of the applicator that extends downwards, from the flat plane or the flat top, into the gripping part. Simply put, there may be no ridge or raised portion or upwardly extending portion within and/or surrounding the flat top of the applicator, i.e. within and/or surrounding a surface of the flat top of the applicator. Furthermore, there may be no indentation or cavity or depression or receptacle formed within the flat top of the applicator i.e. within or in the surface of the flat top of the applicator. In short, the entire application part may be flat. To explain further, the entire top part or surface of the applicator may be flat and forms the application part. By having a flat or planar shape extending over the entire top surface of the applicator simple manufacturing, and easy stacking, e.g. for storing of the system, when the applicator is connected to the container or the dosing unit i.e. as a cap enclosing the dosing unit, can be achieved. Furthermore, the flat-top shape applicator i.e. planar application part extending over the entire top surface of the applicator, ensures easy dispensation of the composition from the applicator onto the skin of the user as compared to applicator having a convex or concave surface or an applicator that has a partial planar top surface and in which such partial planar top surface is bounded by a structure such as a ridge or rim to create a receiving cavity for the composition or which has a receptacle formed therein. Furthermore, the planar application part extending over the entire top surface of the applicator provides more contact area with a skin of the user as compared to applicators having a convex or concave surface or an applicator that has a partial planar top surface bounded by a structure such as a ridge or rim or which has a receptacle formed therein. When a cavity is formed in the application part to receive the composition or when the application part is bound by a ridge or a raised portion, then some amount of composition may be left onto the applicator even after the user attempts to apply the composition onto the skin area. Furthermore, an applicator having partial planar top surface bounded by a structure such as a ridge or rim or which has a receptacle formed therein is not as suitable for spreading function of the composition as is the applicator of the present embodiment having planar application part extending over the entire top surface of the applicator, because such the applicator of the present embodiment would not scrape off or collect the composition from the skin area of the user, when spreading action/function of the applicator is being performed.

The application part of the applicator, the gripping part of the applicator and a part of the applicator which is used for detachably connecting the applicator with the container or the dosing unit may be integrally formed i.e. may be formed as one-part, for example by molding.

In another embodiment of the system, the applicator which is detachably connectable with the container or the dosing unit as explained hereinabove, and particularly at the first locations or the second location, is detachably connectible to the container or the dosing unit via the gripping part. Simply put, the gripping part is detachably connected to the container or the dosing unit to detachably connect the applicator to the container or the dosing unit, generally or particularly at the first locations or the second location.

When the gripping part of the applicator is connected to the container or the dosing unit, an outer surface of the gripping part of the applicator is flush with the container. This makes the system compact and sleek during usage when the applicator is used in a connected state or for storing after use or during transportation.

In one embodiment of the system, the applicator includes flow-arresting means for arresting an accidental flow of the composition flowing in a direction from the application part towards the gripping part. The flow-arresting means may be realized by surface modification such as surface coating and/or surface texturization and/or by one or more flow-arresting structure such as a groove or a rib.

In one embodiment of the system, the gripping part of the applicator includes grip-enhancing means for enhancing a grip of the user at the gripping part. The grip-enhancing means may be realized by surface modification such as surface coating and/or surface texturization and/or by one or more grip-enhancing structure such as a groove or a rib.

In another embodiment of the system, a ratio of a height and a width of the applicator is equal to or less than 3:5. This is advantageous to provide at least a desired minimum separation between the grip of a user at the gripping part and the composition disposed at the application part.

In another embodiment of the system the applicator may be configured for spreading the predetermined amount of the composition on the skin area of the user in a (re)connected state of the applicator and the container or the dosing unit. The (re)connected state may be a state in which the applicator is connected to the container or the dosing unit, for example at the first locations or the second location. The container and the dosing unit may serve as a handle for the applicator, which may facilitate the application of the predetermined dose on the skin. In the connected state, or reconnected state, and with the composition placed, i.e. temporarily coated, on the applicator, a user does not need to directly hold or grip the applicator. The user grips the container and can manipulate or steer the applicator at the skin area, i.e. target area on the user's skin where the composition is desired to be applied by the user, since the applicator is connected to the container directly or is connected to the dosing unit which in turn is connected to the applicator. In short, the applicator may be configured to be connected to the container and the container may be configured to act as an extension handle of the applicator for applying and/or spreading the composition on to the skin area of the user.

Optionally, the applicator, i.e. a surface for example a top flat surface of the applicator configured to receive the composition discharged from the dosing unit, may have a size required for temporarily receiving only the predetermined dose, i.e. the predetermined or metered amount dispensed by the dosing unit. Thus, the system as a whole may have very compact dimensions.

The dosing unit may be a pump dispenser. The pump dispenser may be configured to dispense a predetermined or metered amount in each pump action or for each pump actuation i.e. when the pump is pressed to dispense the composition stored in the container a prefixed or predetermined amount or metered amount of the composition is dispensed out from a dispensing nozzle of the pump dispenser.

According to another embodiment of the system, an outer surface of the applicator, which faces away from the dosing unit in a connected state of the applicator and the dosing unit, for example when the applicator is connected at one of the first locations, may be configured for being temporarily coated with the predetermined dose.

In a further embodiment of the system, an inner surface of the applicator, which faces the dosing unit in a connected state of the applicator and the dosing unit, for example when the applicator is connected at one of the first locations, may be configured for being temporarily coated with the predetermined dose. The applicator may be eversible.

According to another embodiment of the system, the composition may be a liquid, an emulsion, a dispersion, a lotion, a cream, or a paste.

The entire applicator, i.e. including any of its parts comprised in the applicator may be integrally formed i.e. may be formed as one-part, for example by molding, e.g. flow-arresting means and/or grip-enhancing means, etc. may be integrally formed.

Furthermore, it may be noted that it is the top surface of the applicator that is used for receiving the composition and for applying or spreading the composition onto the skin area of the user, therefore making the use of the applicator simpler that if a side surface would be used for the above purposes. When using side surfaces for receiving the composition and for applying or spreading the composition, a chance that the composition may drip off the applicator is high.

According to a second aspect of the present technique, a method for using a system is presented. The system is same as the one explained hereinabove for the first aspect of the present technique. The method includes dispensing, by actuation of the dosing unit, a predetermined amount of composition through the opening of the dosing unit onto the applicator, applying and/or spreading the composition directly from the applicator onto the skin of the user, and detachably connecting the applicator to the container or the dosing unit.

In one embodiment of the method, the method includes washing the applicator after applying and/or spreading the composition.

In a further embodiment the method includes closing the container by detachably connecting the applicator to the container or the dosing unit.

The applying and/or spreading the composition may be performed while the applicator is in a connected state with the dosing unit or the container. Alternatively, the applying and/or spreading the composition may be performed while the applicator is in a disconnected state or detached state from the dosing unit or the container.

According to a third aspect of the present technique, an applicator is presented used in the system of the first aspect. The applicator is used for being temporarily coated or loaded or received with the predetermined amount of the composition and for applying and/or spreading the predetermined amount of the composition on a skin area of a user and used for detachably connecting with the container or the dosing unit. In another embodiment, in addition to the above-mentioned uses of the applicator in the system, the applicator is further used as a closure for the container such that the dosing unit is disposed in an inner space of the applicator when the applicator is detachably connected to the container or to the dosing unit i.e. the applicator is used for closing the container and for housing the dosing unit in the inner space of the applicator when the container is closed using the applicator.

The foregoing and other objects, features, and advantages of the invention are apparent through the following detailed description taking in conjunction with the accompanying drawings in which:

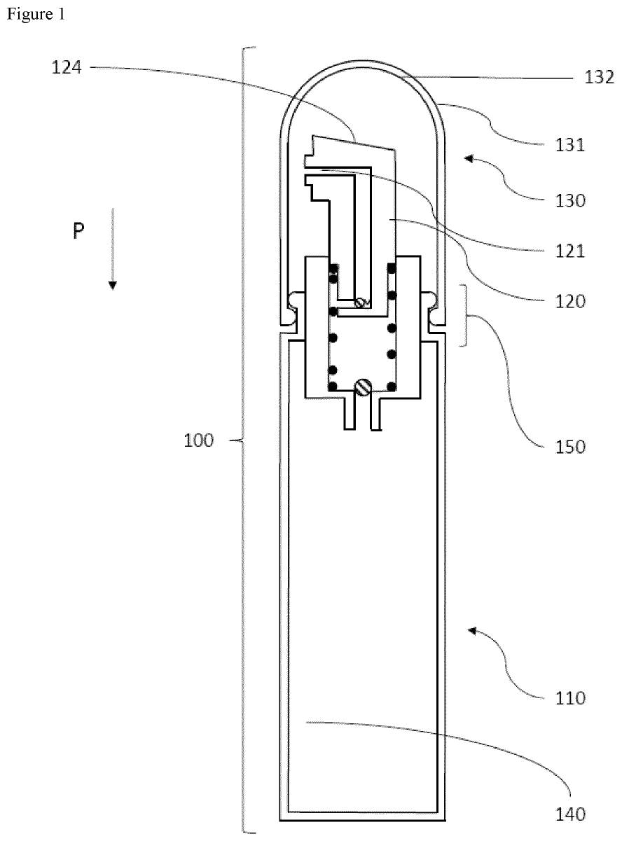

FIG. 1 schematically depicts a first embodiment of a system in a connected state;

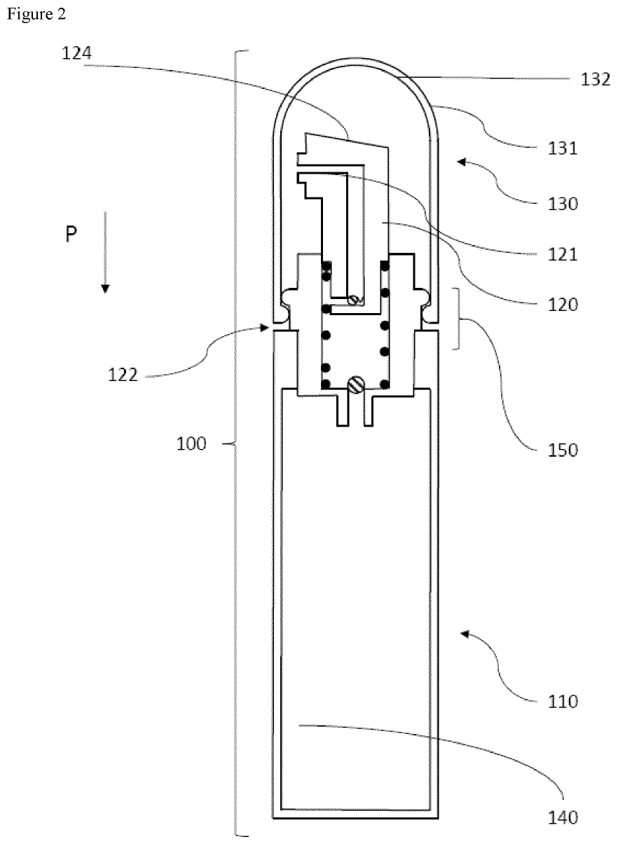

FIG. 2 schematically depicts a second embodiment of the system in a connected state;

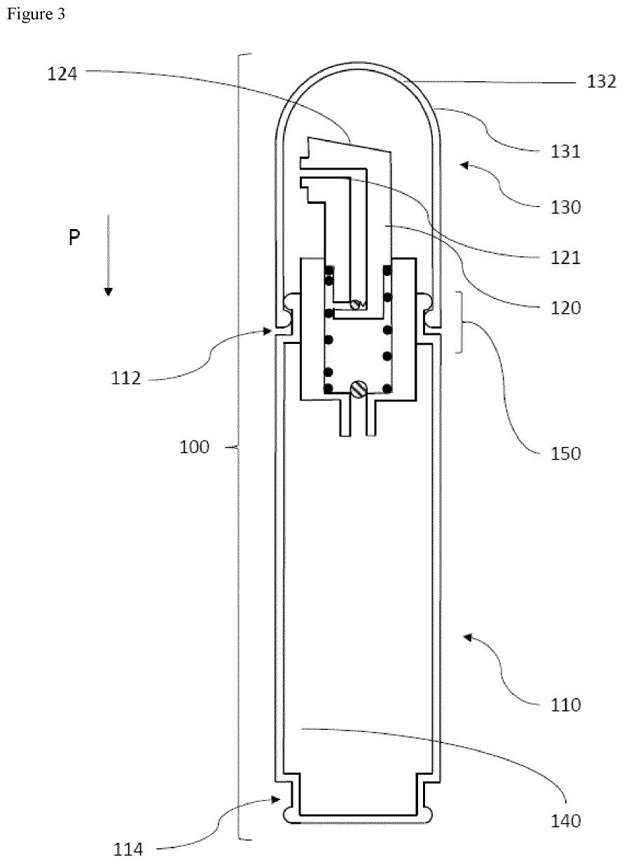

FIG. 3 schematically depicts a third embodiment of the system in a connected state;

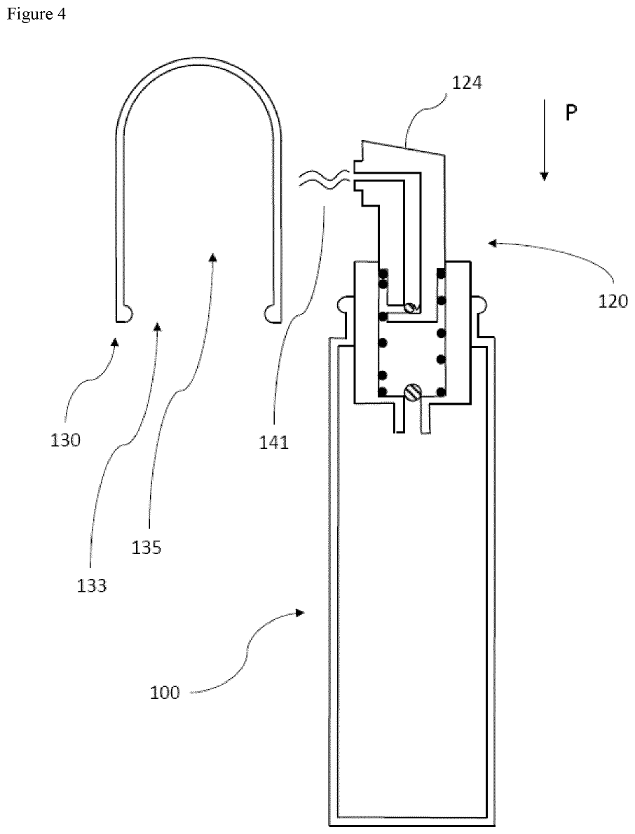

FIG. 4 schematically depicts the first embodiment of the system in a disconnected state;

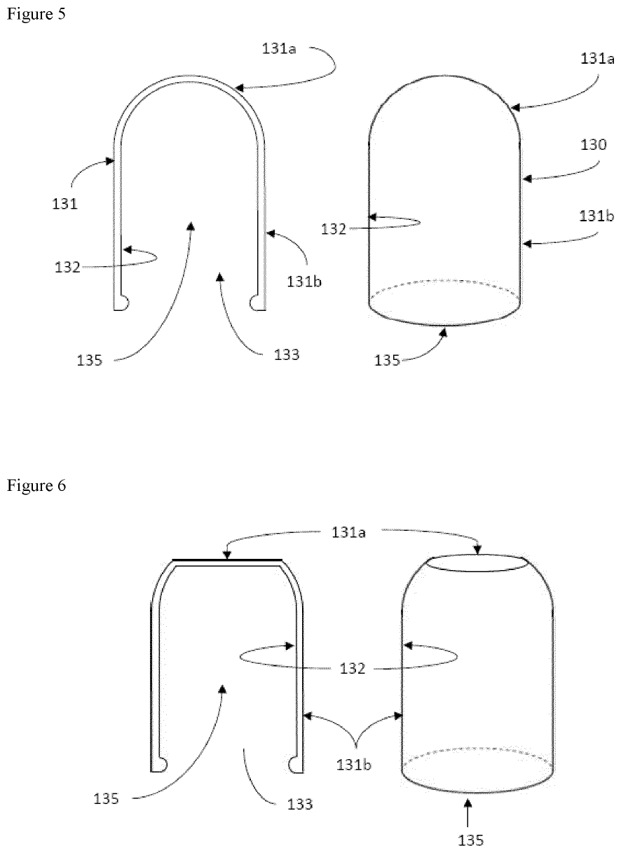

FIG. 5 schematically shows a first embodiment of the applicator depicting a cross-sectional view alongside a perspective view;

FIG. 6 schematically shows a second embodiment of the applicator depicting a cross-sectional view alongside a perspective view;

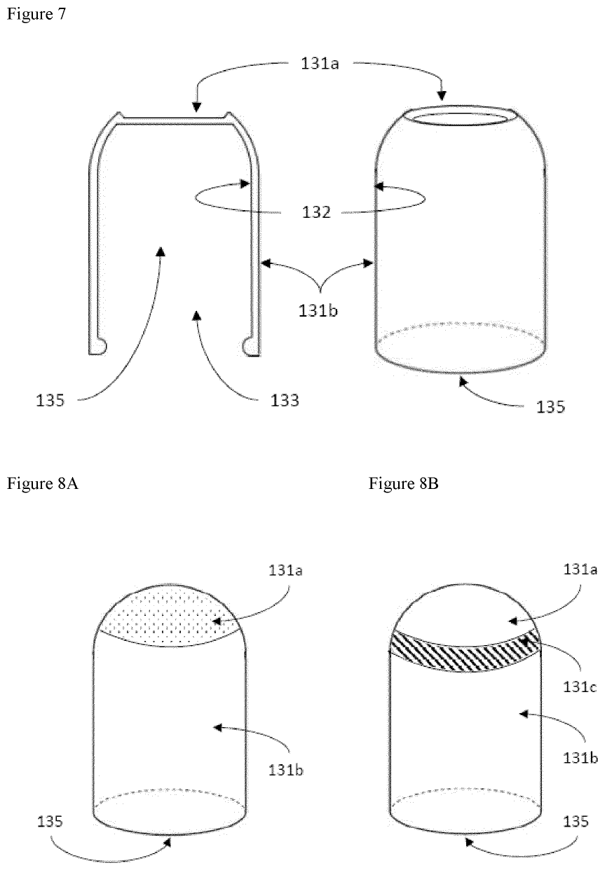

FIG. 7 schematically shows a third embodiment of the applicator depicting a cross-sectional view alongside a perspective view;

FIG. 8A schematically shows an example of modification of a surface of the applicator;

FIG. 8B schematically shows another example of modification of the surface of the applicator;

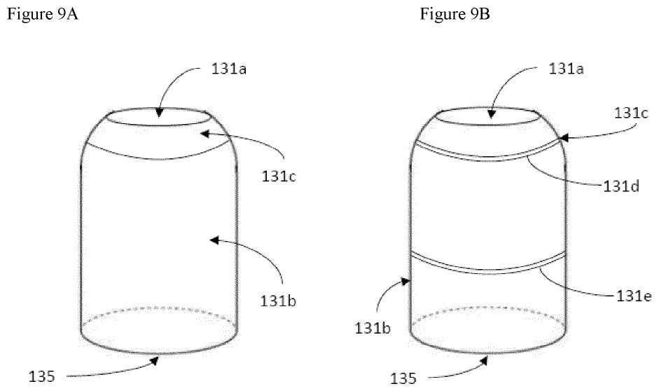

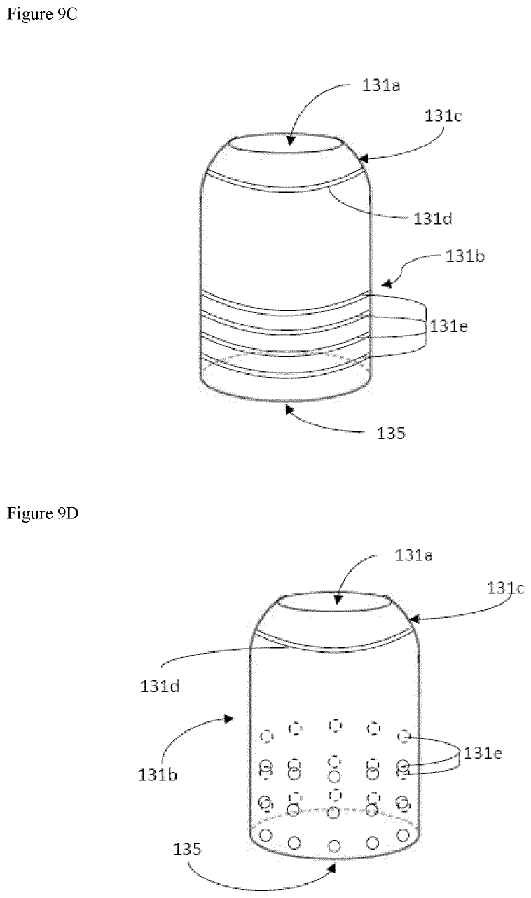

FIGS. 9A-9D schematically show different examples of modification of the surface of the applicator; and

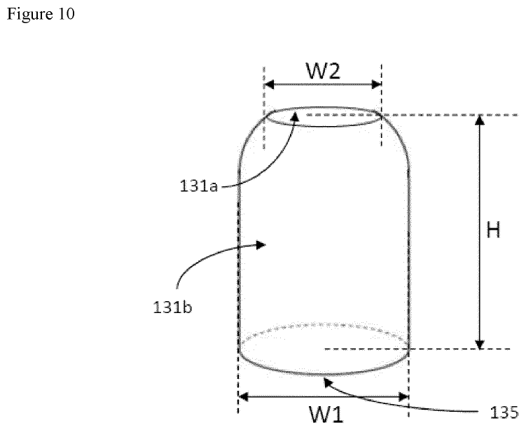

FIG. 10 depicts an embodiment of the applicator depicting exemplary dimensions.

FIGS. 1, 2 and 3 depict a first embodiment, a second embodiment and a third embodiment, respectively, of a system 100 of the present technique. The system 100 may be used for storing a pharmaceutical or cosmetic composition and for applying and/or spreading the composition onto a skin area of a subject, by a user of the system 100. The subject may be the user or another person or being. For sake of simplicity, the present technique has been described for a user who is also the subject, however it should be noted that as stated hereinabove the subject may be different from the user.

As shown in FIGS. 1-3, the system 100 comprises a container 110, a dosing unit 120, and an applicator 130. The system 100 is adapted to receive a pharmaceutical and/or cosmetic composition 140, particularly within the container 110 of the system 100. The composition 140 is stored within the container 110 for being subsequently used. The container 110 may be substantially cylindrical in shape. However, other shapes of the container 110 may also be used, such as, a cuboidal shape e.g. rectangular cuboid. The container 110, generally, may be an elongate body. The container 110 may have the dosing unit 120 disposed at an upper side of the container 110. In the present application, the phrases ‘upper’, ‘lower’, ‘lateral’ and like phrases may indicate positions with reference to an orientation of the container 110 or of the system 100 in which the container 110 or the system 100 is generally stored, for example, when placed on a table top, such as an up-right orientation of the container 110 or the system 100. However, it may be noted that the container 110 or the system 100 may also be placed or stored laying on its side.

The dosing unit 120 is mechanically coupled with the container 110. The dosing unit 120 may be mechanically coupled with the container 110 specially when the composition 140 is in the container 110. Once the composition 140 is inside the container 110, the dosing unit 120 may be fixedly attached to the container 110 or may be detachably attached to the container 110. When fixedly attached to the container 110, a user may not be able to separate the dosing unit 120 from the container 110 thereby obviating unintended spillage of the composition 140. When detachably attached to the container 110, the dosing unit 120 may be separated from the container 110, for example to fill or refill the container 110 with the composition 140 or with another product or composition.

In particular, the dosing unit 120 may close an opening of the container 110 such that the pharmaceutical and/or cosmetic composition 140 is confined within the container 110, unless purposively expelled or dispensed by a user by operation of the dosing unit 120. A joint between the container 110 and the dosing unit 120 may be sealed such that the composition 140 does not spill or seep out of the container 110 when the system 100 or the container 110 are tilted or laid laterally on a surface, or even when positioned in upside down orientation. The container 110 with the dosing unit 120 may generally be configured as an airless dispenser that allows the storage and dispensing of a viscous composition, for example a gel-based composition or a paste.

The dosing unit 120 includes an opening 121 through which the composition 140 is dispensed, when the dosing unit 120 is operated by a user. The dosing unit 120 may be operated by a user in variety of ways, for example by pressing an actuation surface 124 of the dosing unit 120, for example in a downward direction shown in FIGS. 1-4 by the arrow marked with reference sign ‘P’. Alternatively, the dosing unit 120 may be operated for example by squeezing a lateral side of the dosing unit 120 or for example by squeezing a lateral side of the container 110. The dosing unit 120 may be operable to dispense the composition 140 through the opening 121 while the dosing unit 120 is mechanically coupled to the container 110. It may be noted that the modes of operating the dosing unit 120 are not limited to the above-mentioned examples and the dosing unit 120 may have any mode of operation which enables a user to dispense the composition 140 through the opening 121, in predetermined amount.

The applicator 130 is configured to be temporarily coated or wetted with the predetermined amount of the composition 140 and then used for applying and/or spreading the predetermined amount of the composition on a skin area of a user. The user can grip or hold the applicator 130 and manipulate the applicator 130 to apply and/or spread the composition 140 at a desired skin area of the user.

The applicator 130 may be configured to be mechanically coupled with the container 110 as shown in FIGS. 1 and 3, while the dosing unit 120 is mechanically coupled to the container 110. The applicator 130 is detachably or removably coupled with the container 110 i.e. a user may detach or remove or decouple the mechanically coupled applicator 130 of FIGS. 1 and 3 from the container 110 as shown in FIG. 4. When decoupling or removing the applicator 130 from the container 110, the dosing unit 120 remains mechanically coupled to the container 110.

Alternatively, as shown in FIG. 2 the applicator 130 may be configured to be mechanically coupled with the dosing unit 120, while the dosing unit 120 is mechanically coupled to the container 110. The applicator 130 is detachably or removably coupled with the dosing unit 120 i.e. a user may detach or remove or decouple (not shown) the mechanically coupled applicator 130 of FIG. 2 from the dosing unit 120. When decoupling or removing the applicator 130 from the dosing unit 120, the dosing unit 120 remains mechanically coupled to the container 110.

According to the first, the second and the third embodiments of the system 100 shown in FIGS. 1 to 4, a first coupling 150, i.e. the detachable connection between the applicator 130 and the dosing unit 120 (as shown in FIG. 2) or the detachable connection between the applicator 130 and the container 110 (as shown in FIGS. 1, 3 and 4) may be achieved by means of a snap-on connection. Generally, the first coupling 150 may be, but not limited to, a plug-in connection, a snap-on connection, a screw and thread connection, a friction-fit connection and a combination thereof. The applicator 130 may be realized as a closure such that when the applicator 130 is in the coupled state i.e. when the applicator 130 is coupled to the container 110 (as shown in FIGS. 1 and 3) or coupled to the dosing unit 120 (as shown in FIG. 2), by using the coupling 150, at least a part of the dosing unit 120 having the opening 121 is disposed in an inner space 135 of the applicator 130.

The applicator 130 may be detachably connectible to the container 110 at a first location 112 of the container 110 or at a first location 122 of the dosing unit 120, as explained hereinabove with reference to FIGS. 1 and 3 and with reference to FIG. 2, respectively. When connected at the first location 112 or the first location 122, the first coupling 150 is formed. When connected at the first location 112 of the container 110 or at the first location 122 of the dosing unit 120, the applicator 130 functions as a closure of the container 110 and/or the dosing unit 120.

When connected at the first location 112 of the container 110 or at the first location 122 of the dosing unit 120, the applicator 130 may also function to spread the predetermined amount 141 of the composition 140 on the skin area of a user. For using the applicator 130 to spread the predetermined amount 141 of the composition 140 on the skin area while being connected at the first location 112 of the container 110 or at the first location 122 of the dosing unit 120, the predetermined amount of the composition 140 is dispensed from the opening 121, by actuation of the dosing unit 120, and disposed (i.e. temporarily coated) on the applicator 130 while the applicator 130 is detached from the container 110 and/or the dosing unit 120. Once the predetermined amount 141 of the composition 140 is temporarily coated on the applicator 130, the applicator 130 is connected (i.e. reconnected) at the first location 112, 122 and can be used to spread and/or spread the predetermined amount 141 of the composition 140 on the skin area, while remaining connected at the first location 112, 122.

In another embodiment, the applicator 130 may be detachably connectible to the container 110 at a second location 114 of the container 110, independent of being connectible at the first locations 112, 122, i.e. the applicator 130 may or may not be configured to be detachably connectible to the first locations 112, 122.

Optionally, the applicator 130 may be detachably connectible to the container 110 at the second location 114 of the container 110, in addition to being detachably connectible at the first location 112 of the container 110 or at the first location 122 of the dosing unit 120, for example as shown in FIG. 3. When connected at the second location 114 of the container 110, the applicator 130 and the container 110 form a second coupling (not shown).

Generally, the second coupling may be, but not limited to, a plug-in connection, a snap-on connection, a screw and thread connection, a friction-fit connection and a combination thereof.

When connected at the second location 114 of the container 110, the applicator 130 may function to spread the predetermined amount 141 of the composition 140 on the skin area of a subject or user. For using the applicator 130 to spread the predetermined amount 141 of the composition 140 on the skin area while being connected at the second location 114 of the container 110, the predetermined amount of the composition 140 is dispensed from the opening 121, by actuation of the dosing unit 120, and disposed (i.e. temporarily coated) on the applicator 130 while the applicator 130 is detached from the container 110 and/or the dosing unit 120. Once the predetermined amount of the composition 140 is temporarily coated on the applicator 130, the applicator 130 is connected at the second location 114 and can be used to spread the predetermined amount 141 of the composition 140 on the skin area, while remaining connected at the second location 114.

When the applicator 130 is used to spread the predetermined amount 141 of the composition 140 on the skin area, while being at the first location 112, 122 or while being connected at the second location 114, the container 110 acts as an extension handle for the applicator 130. Thus, a larger separation is realized between the composition 140 temporarily coated on the applicator 130 and the fingers of the user gripping the extension handle. Furthermore, due to the extension handle hard to reach parts of the body, such as back of the user, may be reached more easily.

It may be noted that the applicator 130 can be used to apply and/or spread the composition 140 onto the skin area while being unattached or disconnected from the container 110 or the dosing unit 120.

The dosing unit 120 is configured for dispensing the predetermined amount 141 (shown in FIG. 4) of the composition 140 through the opening 121 of the dosing unit 120, for each operation or actuation of the dosing unit 120. For example, a user of the system 100 may press an actuator i.e. for example the actuation surface 124, of the dosing unit 120 in the direction P (along the longitudinal direction of the container 110) for dispensing the predetermined amount 141 out of the container 110 through the opening 121 of the dosing unit 120. The ‘predetermined amount’ as used herein includes a fixed quantity or a metered quantity of the composition 140. For example, on one actuation or one operation of the dosing unit 120, such as a single press of the actuation surface 124 in the direction P, a fixed or metered amount of the composition 140 is dispensed through the opening 121. The ‘predetermined amount’, dispensed through the opening 121 per actuation of the dosing unit 120, may be, but not limited to, between 0.05 ml (milliliter) and 1.00 ml of the composition 140, particularly between 0.1 ml and 0.75 ml of the composition 140, and more particularly between 0.2 ml and 0.5 ml of the composition 140. In an embodiment of the system 100, the predetermined amount 141 dispensed through the opening 121 per actuation of the dosing unit 120 is AA 0.3 ml of the composition 140.

The predetermined amount 141 of the pharmaceutical composition may be used to coat a part or a region of an outer surface 131 and/or a part or a region of an inner surface 132 of the applicator 130. In case of a liquid pharmaceutical composition, the region of the inner and/or the outer surface 131, 132 of the applicator 130 may in particular be wetted with the predetermined amount 141 of the composition 140. The region of the inner and/or the outer surface 131, 132 of the applicator 130 coated or wetted with the composition 140 is configured to receive the composition 140 dispensed from the opening 121 of the dosing unit 120, when the dosing unit 120 is actuated.

Hereinafter, further detail of the applicator 130 have been described with reference to FIGS. 5 to 10. It may be noted that applicator 130 defined hereinafter with reference to FIGS. 5 to 10 may be the applicator 130 detachably connectable with the container 110, as shown in FIGS. 1 and 3, or the applicator 130 detachably connectable with dosing unit 120, as shown in FIG. 2.

The applicator 130 comprises an application part 131a and a gripping part 131b. The application part 131a is configured to receive the composition 140, preferably predetermined amount 141 of the composition 140, from the opening 121, when the dosing unit 120 is actuated. The application part 131a, on receiving the composition 140, gets temporarily coated by the composition 140. The gripping part 131b is configured to be gripped or held by a user. The gripping part 131b is not intended for receiving the composition 140 from the dosing unit 120. The application part 131a and the gripping part 131b may be formed monolithically, or alternatively the application part 131a and the gripping part 131b may be formed separately and then assembled with each other to realize the applicator 130.

The application part 131a and the gripping part 131b may be formed of same material, for example both parts i.e. the application part 131a and gripping part 131b may be formed of a polymer such as polypropylene (PP). Alternatively, the application part 131a and the gripping part 131b may be formed of different materials, for example the application part 131a may be formed of a polymer such as polypropylene (PP), whereas the gripping part 131b may be formed of another polymer such as rubber.

In an embodiment, the application part 131a is non-deformable. The phrase ‘non-deformable’ as used in the present technique means a structure that does not get deformed by intended usage or that does need to be deformed for intended usage. For example, the application part 131a is non-deformable as opposed to conventionally known brush or foam applicator devices. In addition to or as an alternative to the non-deformable application part 131a, in another embodiment of the system 100, the application part 131a is non-porous. The phrase ‘non-porous’ as used in the present technique means a structure such that the composition 140 does not penetrate or impregnate the application part 131a when the composition 140 is dispensed by the dosing unit 120 onto the application part 131a, simply put the composition 140 stays atop the surface of the application part 131a before being transferred to the skin area of the user.

The application part 131a of the applicator 130 is configured to come into contact with the skin area of the user where the composition 140 is applied and/or spread and thus the application part 131a may be formed devoid of sharp edges or surface structures that may irritate or affect the user's skin. The gripping part 131b of the applicator 130 is configured to come into contact with fingers of the user for purpose of holding and manipulating the applicator 130.

As shown in FIG. 5, in an embodiment the application part 131a may have a convex shape to facilitate spreading the predetermined amount 141 of the pharmaceutical composition on the skin area. The convex shape is especially advantageous to spread the composition 140 onto focused areas or ‘spots’ (for example on a pimple or a boil on the skin) or skin of folded regions of the body such as in-between digits (fingers or toes) of hands or feet.

Alternatively, in another embodiment as shown in FIG. 6, the application part 131a may have a flat-top shape i.e. is flat or planar, to facilitate spreading the predetermined amount 141 of the pharmaceutical composition on the skin area. The flat top shape is especially advantageous to spread the composition 140 onto flat or curved areas that are readily accessible areas of the body such as forehead of the user.

In yet another embodiment as shown in FIG. 7, the application part 131a may have a concave shape or a depression to facilitate spreading the predetermined amount 141 of the pharmaceutical composition on the skin area. The concave shape or a depression is especially advantageous to hold and spread compositions having less viscosity, or when the predetermined amount is large. In one embodiment, the depression is between about 0.05 cm (centimeter) to about 0.5 cm in depth. In another embodiment the depression is between about 0.05 cm to about 0.4 cm in depth. In yet another embodiment the depression is between about 0.1 cm to about 0.2 cm in depth.

FIGS. 8A and 8B show different surface modifications that may be implemented in any of the embodiments of the applicator 130 shown in FIGS. 5-7. Although in FIGS. 8A and 8B only the applicator 130 having a convex shaped application part 131a as also shown in FIG. 5 is depicted, it may be appreciated by one skilled in the art that one or more of the same surface modifications may be implemented in the applicators 130 depicted in FIGS. 6 and 7.

The surface of the applicator 130 in the application part 131a may be different from the surface of the applicator 130 in the gripping part 131b, as shown in FIG. 8A. For example, the surface of the applicator 130 in the application part 131a may be smooth to facilitate transfer of the composition 140 from the application part 131a onto the skin area of the user, whereas the surface of the applicator 130 in the gripping part 131b may be rough, at least compared to the surface of applicator 130 in the application part 131a, to facilitate non-slip grip by the user.

Optionally, as shown in FIG. 8B, a boundary or a boundary region 131c may be present in the applicator 130 between the surfaces of the application part 131a and the gripping part 131b. The surface of the boundary region 131c may comprise flow arresting means 131d (shown in FIGS. 9B-9D) that arrests or stops a flow of the composition 140 in case the composition spills or flows out of the application part 131a. The flow arresting means may be a coating on the surface 131 of the applicator 130 or a texturing of the surface 131 of the applicator 130 that resists or inhibits flow of the composition 140 from the application part 131a towards the gripping part 131b.

FIGS. 9A to 9D show surface modifications that may be implemented in any of the embodiments of the applicator 130 shown in FIGS. 5-7. Although in FIGS. 9A to 9D only the applicator 130 having a flat-top shape application part 131a as also shown in FIG. 6 is depicted, it may be appreciated by one skilled in the art that one or more of the same surface modifications may be implemented in any one of the applicators 130 depicted in FIGS. 5 and 7.

FIG. 9A shows the boundary or boundary region 131c formed in the applicator 130 between the surfaces of the application part 131a and the gripping part 131b. The surface of the boundary region 131c may comprise flow arresting means 131d (not shown in FIG. 9A) that arrests or stops a flow of the composition 140 in case the composition spills or flows out of the application part 131a, for example the boundary region 131c may comprise a flow-arresting structure 131d such as a lateral groove or a lateral ridge, that arrests or stops a flow of the composition 140 from the application part 131a towards the gripping part 131b, as shown in FIG. 9B. The flow-arresting structure 131d extends on the surface 131 of the applicator 130 such that the flow-arresting structure 131d surrounds or encircles the surface of the application part 131a i.e. the surface of the application part 131a which is intended to receive the composition 140 from the dosing unit 120. In one embodiment, the flow-arresting structure 131d extends on the surface 131 of the applicator 130 such that the flow-arresting structure 131d completely surrounds or completely encircles the surface of the application part 131a.

In another embodiment, the applicator 130 may include grip-enhancing means 131e, independent of the flow-arresting means 131d, i.e. flow-arresting means 131d may or may not be present when the grip-enhancing means 131e is present in the applicator 130.

In yet another embodiment, in addition to the flow-arresting structure 131d, the applicator 130 includes, in the gripping part 131b, the grip-enhancing means 131e that may be realized as one or more grip-enhancing structures 131e. FIG. 9B shows one such grip-enhancing structure 131e. FIG. 9C shows a plurality of such grip-enhancing structures 131e. The grip-enhancing structure 131e enhances or facilitates a grip of the user such that a risk of the applicator 130 slipping from the user's hand during usage is reduced. The grip-enhancing structure 131e as shown in FIGS. 9B and 9C may be formed as a groove or a ridge, extending laterally. It may be noted that the groove or ridge that is formed as the grip-enhancing means 131e may be not be extending laterally and may instead extend in another direction such as vertically. In another embodiment as shown in FIG. 9D, the grip-enhancing structure 131e may be formed as dimples or protuberances on the surface of the gripping part 131b of the applicator 130.

FIG. 10 schematically presents a height H, a base diameter W1 and a top diameter W2 of the applicator 130. The height H of the applicator 130 is a length of the applicator 130 along a longitudinal axis (not shown) of the applicator 130, for example a distance between an opening 133 of the applicator 130, the opening 133 which is used to mechanically couple the applicator 130 and the dosing unit 120 and/or the container 110 at the respective first locations 122, 112. The base diameter W1 is the outer diameter of the opening 133 i.e. diameter including the lateral walls of the applicator 130. The top diameter W2 is the diameter of the top surface of the applicator 130, for flat-top shape and concave shaped applicator 130 as shown in FIGS. 6 and 7.

In one embodiment, for any of the applicators 130 of FIG. 5, 6 or 7, the ratio W1:H is equal to or less than 3:5, i.e. for example 1:2, 3:7, 3:8, and 1:3. Additionally, for the applicator 130 of FIG. 6 or 7, the ratio of W1:W2 is equal to or less than 2:3, i.e. for example 1:2, 2:5, 1:3, and 2:7. In an exemplary embodiment, as shown in FIG. 10, the ratio W1:W2:H is about 3:1.5:5, which is advantageous in providing a substantial space for flat-top and also ensures enough space for gripping and a substantial separation between the flat-top where the composition 140 is disposed and the fingers of the user gripping the applicator 130.

In another embodiment of the system 100, the applicator or a part of the applicator is formed of a flexible material, and in this embodiment an inner surface 132 may be used for spreading the composition 140 onto the skin area of the user or another subject. The flexible material allows everting the applicator so that the predetermined amount 141 of the pharmaceutical composition 140 can be applied and/or spread onto the skin area. In other words, the inner surface with the predetermined amount 141 of the pharmaceutical composition is reverted inside-out allowing an application of the composition on the user's skin.

In particular, parts of the outer surface 131 and/or the inner surface 132 of the applicator 130 that are comprised in the application part 131a may be free of edges and/or corners to avoid any irritation of the skin area coming in contact with such edges and/or corners of the outer surface 131 and/or the inner surface 132 of the applicator.

The outer surface 131 and/or the inner surface 132 of the applicator 130 may be hydrophobic or hydrophilic depending on the pharmaceutical and/or cosmetic composition. Preferably the surface of the applicator 130 used for receiving the composition is hydrophobic. A suitable material of the applicator 130 is polypropylene (PP), however, also other polymeric materials known to the skilled person in the technical field of applicator systems can be used. The quality most preferably complies with Ph. Eur. § 3.1.3 Polyolefins and § 3.1.6 Polypropylenes for containers and closures for parenteral and ophthalmic preparations.

The outer surface 131 and/or the inner surface 132 of the applicator 130 more preferably is roughened in order to avoid a slipping or gliding of the composition 140. A slipping or gliding is particularly problematic if the composition is an oil-in-water emulsion.

The pharmaceutical and/or cosmetic composition 140 may be an oil-in-water emulsion (O/W) comprising a glycopyrronium salt (GP salt) and an emulsifier system. Preferably the emulsifier system comprises at least one macrogol glycerol fatty acid ester, at least one glycerol fatty acid ester and at least one fatty alcohol. Such an emulsion may be used as a medicament, in particular for treating and preventing diseases in conjunction with excessive sweating (hyperhidrosis). In a non-therapeutic (cosmetic) use, such an oil-in-water emulsion for topical application may be used on the skin of a mammal in order to reduce sweating. The composition 140 may contain pharmaceutically and cosmetically acceptable excipients known to the skilled person. The form of the composition 140 is not particularly limited, however, it preferably is a lotion or cream. Particularly preferred embodiments of the composition 140 are disclosed in the co-pending applications EP 17 181 684 and PCT/EP2018/069273 being incorporated herein by reference. For glycopyrronium salts (GP salts) the application to the skin of the user in a well-defined way is particularly important. The anticholinergic effect of GP may result in a toxicity or undesired pharmacological effects, such as skin irritation or blotching, for the user if administered in high amounts or to unwanted areas of the body. Thus, the system 100 of the invention allows to significantly minimize the risk of a toxic or undesired effect such as an unintended oral administration, accidental ingestion, accidental contamination of body parts such as eyes or ear canal, accidental insufflation, pulmonary aspiration, etc. through composition contaminated hands.

In one embodiment the system 100 includes the composition 140, for example a composition comprising a glycopyrronium salt (GP salt) and an emulsifier system is stored or filled within the container 110.

The system 100 is useful for a topical dermal composition. In one embodiment the system 100 is used for storing, dispensing, and applying and/or spreading of a topical composition for medicinal and/or cosmetic use.

In one embodiment of the system 100, the applicator 130 is configured to be temporarily coated with the predetermined amount 141 of the composition 140, and further configured for spreading the predetermined amount 141 of the composition 140 on a skin area of a user, and also configured to be used as a closure for the container 110 and/or the dosing unit 120, by being mechanically coupled, at the first location 112, 122 of the container 110 and/or the dosing unit 120, respectively. Thus, the applicator 130 may be used as a closure for the container 110 and/or the dosing unit 120 and used for being temporarily coated with the predetermined amount 141 of the composition 140 and for spreading the predetermined amount 141 of the composition 140 on a skin area of a user.

The present technique envisages the applicator 130 used in the system 100, wherein the system is as explained hereinabove with reference to FIGS. 1-10. The system 100 includes a container 110 for receiving a pharmaceutical and/or cosmetic composition 140; a dosing unit 120 mechanically coupled with the container 110 for dispensing a predetermined amount 141 of the composition 140 from the container 110 through an opening 121 of the dosing unit 120; and an applicator 130 configured to be temporarily coated, in particular wetted, with the predetermined amount 141 of the composition 140 and configured for spreading the predetermined amount 141 of the composition 140 on a skin area of a user and wherein the applicator 130 is detachably connectable with the container 110 or the dosing unit 120, and wherein the applicator 130 is used for being temporarily coated, in particular wetted, with the predetermined amount 141 of the composition 140 and for spreading the predetermined amount 141 of the composition 140 on a skin area of a user and for detachably connecting with the container 110 or the dosing unit 120. In another embodiment, in addition to the above-mentioned use of the applicator 130 in the system 100, the applicator 130 is used as a closure for the container 110 such that the dosing unit 120 is disposed in an inner space 135 of the applicator 130 when the applicator 130 is connected to the container 110 or the dosing unit 120 i.e. the applicator 130 is used for closing the container 110 and housing the dosing unit 120 in the inner space 135 of the applicator 130 when the container 110 is closed using the applicator 130.

### LIST OF REFERENCE SIGNS

100 system
110 container
112 first location of the container
114 second location of the container
120 dosing unit
121 opening
122 first location of the dosing unit
124 actuation surface of the dosing unit
130 applicator
131 outer surface of the applicator
131a application part
131b gripping part
131c boundary region
131d flow-arresting means
131e grip-enhancing means
132 inner surface of the applicator
133 opening of the applicator
135 inner space of the applicator
140 pharmaceutical and/or cosmetic composition
141 predetermined amount of the composition
150 snap-on connection

1. A system comprising:
a container configured to store a pharmaceutical and/or cosmetic composition;
a dosing unit mechanically coupled with the container and configured to dispense a predetermined amount of the composition from the container through an opening of the dosing unit; and
an applicator;
wherein
the applicator is configured to be temporarily loaded with the predetermined amount of the composition;
wherein the applicator is configured to apply and/or spread the predetermined amount of the composition on a skin area of a user; and
wherein the applicator is detachably connectable with the container or the dosing unit.

2. The system according to claim 1, wherein the applicator is configured to be a closure for the container such that the dosing unit is disposed in an inner space of the applicator, when the applicator is connected at a first location of the container or at a first location of the dosing unit.

3. The system according to claim 1, wherein the applicator, when connected at the first location of the container or at the first location of the dosing unit, is configured to seal the opening of the dosing unit.

4. The system according to claim 1, wherein the applicator is detachably connectible to the container at a second location of the container such that the container forms an extension handle of the applicator.

5. The system according to claim 1, wherein the applicator comprises:
a gripping part configured to be gripped by the user, and
an application part configured to receive the composition dispensed through the opening of the dosing unit and to apply and/or spread on the skin of the user the composition so received,
wherein the application part has one of convex shape, a flat shape and a concave shape.

6. The system according to claim 5, wherein the applicator is detachably connectable with the container or the dosing unit by connecting the gripping part of the applicator to the container or the dosing unit.

7. The system according to claim 6, wherein an outer surface of the gripping part of the applicator is configured to be flush with the container when the gripping part of the applicator is connected to the container or the dosing unit.

8. The system according to claim 5, wherein the applicator comprises flow-arresting means configured to arrest a flow of the composition flowing in a direction from the application part towards the gripping part.

9. The system according to claim 5, wherein the gripping part comprises grip-enhancing means configured to enhance a grip of the user at the gripping part.

10. The system according to claim 1, wherein a ratio of a width (W1) and a height (H) of the applicator is equal to or less than 3:5.

11. The system according to claim 1, wherein the applicator is configured for spreading the predetermined amount of the composition on the skin area of the user in a connected state of the applicator and the container or the dosing unit.

12. The system according to claim 1, wherein an outer surface of the applicator, which faces away from the dosing unit in a connected state of the applicator and the dosing unit or the container, is configured for being temporarily loaded with the predetermined dose.

13. The system according to claim 1, wherein an inner surface of the applicator, which faces the dosing unit in a connected state of the applicator and the dosing unit or the container, is configured for being temporarily loaded with the predetermined dose.

14. The system according to claim 1, wherein the composition is a pharmaceutical and/or cosmetic composition in the form of an oil-in-water emulsion comprising a glycopyrronium salt and an emulsifier system.

15. A method for using a system according to claim 1, the method comprising:
detaching, from the container or the dosing unit, the applicator connected with the container or the dosing unit to expose the dosing unit;
dispensing, onto the applicator, a predetermined amount of the composition from the container through an opening of the dosing unit by actuating the dosing unit, to load the applicator with the predetermined amount of the composition;
applying and/or spreading the predetermined amount of the composition loaded on the applicator, from the applicator directly onto a skin area of a user by contacting the applicator directly with the skin area of the user; and
connecting the applicator with the container or the dosing unit such that the dosing unit is disposed in an inner space of the applicator.
